(19) United States
(12) Patent Application Publication
(10) Pub. No.: US 2006/0046274 A1
(43) Pub. Date: Mar. 2, 2006

(54) Inhibin-A: a marker for differentiation, diagnosing and screening abnormal pregnancies

(75) Inventors: Shimon Segal, Brookline, MA (US); Benjamin Rivnay, Newton, MA (US)

Correspondence Address: PALMER & DODGE, LLP;KATHLEEN M. WILLIAMS, 111 HUNTINGTON AVENUE, BOSTON, MA, 02199, US

(73) Assignee: Repromedix Corporation and Shimon Segal

(21) Appl. No.: 10/930,429

(22) Filed: Aug. 31, 2004

(51) Int. Cl. G01N 33/542; G01N 33/53

(52) U.S. Cl. 435/7.9

(57) ABSTRACT

The present invention relates to a method, based on the hormone Inhibin-A, of diagnosing, screening and differentiating between normal pregnancies and abnormal pregnancies. The method can be used to differentiate between viable and non-viable pregnancies. Particularly, Inhibin-A can also be used in a method and kit for the diagnosis of ectopic pregnancy.

### BACKGROUND OF THE INVENTION

The present invention relates to a method of differentiating, diagnosing and screening between normal and abnormal pregnancies, particularly in the first trimester of pregnancy. Principally, the present invention relates to differentiating and diagnosing a viable or non-viable pregnancy abnormality by determining the level of Inhibin-A in a patient. The Inhibin-A marker can be used to screen pregnant women for pregnancy abnormalities and to differentiate among abnormal pregnancies. The patient may or may not manifest clinical symptoms of a pregnancy abnormality prior to using the inventive method and kit. The marker is particularly effective in the diagnosis and screening of an ectopic pregnancy.

A well-known example of a pregnancy abnormality is an ectopic pregnancy (EP). It is a life threatening medical condition afflicting 2.72% of pregnancies. EP occurs when the fertilized egg implants in the fallopian tubes. As the embryo grows, it may cause pain and bleeding. If EP is not treated quickly enough, the tube can rupture and cause abdominal bleeding. In extreme cases, EP can lead to maternal death. Fifteen percent of all maternal deaths have been attributed to complications of EP. There is a significantly increased risk of EP for African American and other minorities. Notably, women who had an ectopic pregnancy have an increased risk for a subsequent EP.

Diagnosis of EP commonly occurs on week 5-10 of gestation and is complicated by a wide spectrum of clinical presentations. To date, the most assertive determination of EP is based on the absence of an embryo by ultrasound with a βhCG level >1500 IU/ml, or if βhCG levels are lower than 1500 IU/ml but fail to increase by 60% within 48 hrs. Earlier studies have attempted to harness the measurement of various proteins, or other endocrine markers to a rigorous clinical diagnostic protocol for EP evaluation or confirmation. These efforts did not materialize due to various conclusions from several studies, such as, an overlap between the ranges of analyte levels in intra-vs. extra-uterine pregnancies, lack of correlation between the abnormal levels of the analyte and the clinical presentation of the patient, lack of prognostic or diagnostic value to a single measurement (as in the case of βhCG), or conflicting reports on the same analyte. Thus, there is no effective marker that clearly diagnoses or screens the state of a pregnancy abnormality, such as EP, even upon manifestation of its clinical symptoms.

Inhibin-A is a 31 kD disulfide linked heterodimeric protein hormone secreted by the luteal phase ovary and by the placenta. In normal pregnant women, serum Inhibin-A concentration starts at the range of normal luteal phase. It subsequently climbs to a higher level by week 8 and remains stable with a small decrease until week 27. The concentration then rises throughout the third trimester of the pregnancy.

Tests for Inhibin-A have been previously developed. Groome et al disclose immunoassays for the detection of Inhibin and its subunits in biological fluids. J Immunological Methods 1993:165: pp. 167-176. Significant levels of Inhibin immunoreactivity was found in serum from women undergoing ovulation induction and in normal women, as opposed to postmenopausal women. Although the authors proposed that the Inhibin immunoassays may have potential uses, there is no discussion on using an Inhibin immunoassay to diagnose an abnormal pregnancy, such as an ectopic pregnancy.

Inhibin-A concentrations in EP have been previously measured. Seifer et al have noted that, in a study on natural cycle pregnancies, serum Inhibin levels are lower in EP than in intrauterine spontaneously conceived pregnancies (IUP). Fertility and Sterility, Vol. 66, No. 3, March 1996. The differences between EP and IUP patients in Seifer et al. were greater for progesterone levels and total Inhibin than for the dimeric Inhibin-A levels. Moreover, the ranges of Inhibin-A levels within the EP and IUP groups partially overlapped. Thus, Seifer et al. concluded that total Inhibin was a better marker than dimeric Inhibin for diagnosing EP and for differentiating between EP and IUP.

Yohkaichiya et al. published results of a study on IVF patients (ovarian hyperstimulated pregnancies) wherein serum immunoreactive Inhibin concentrations were found to be elevated in IVF patients during the first trimester when compared with those in a normal pregnancy. Fertility and Sterility, Vol. 59, No. 5, March 1993. However, Yohkaichiya et al. concluded that the measurement of Inhibin-A alone would be an unlikely marker in differentiating among normal and abnormal pregnancies.

U.S. Pat. No. 5,906,944 describes a method for antenatal screening for an abnormality in a fetus using a bodily fluid from the subject. The procedure involves measuring a marker at different periods of gestation. The two determinations of the marker are separated in time by at least three weeks. At a first stage of gestation, the median level of the marker differs by less than 20% between pregnancies affected and unaffected by the abnormality. At a second stage of gestation, the difference between pregnancies affected and unaffected by the abnormality is 50%. Normalized concentration values of the markers for both stages are determined. An abnormal fetal abnormality may be diagnosed after obtaining the two samples from the patient over the span of at least three weeks and analyzing the results of the normalized concentration values.

None of these solve the problems of determining an abnormal pregnancy, such as an ectopic pregnancy, in a female patient who may or may not manifest symptoms of a pregnancy abnormality.

Thus, an object of this invention is to provide a means for diagnosing, differentiating, screening and/or managing an abnormal pregnancy, such as an ectopic pregnancy, in a female patient using a marker that does not require measuring at different periods of gestation or obtaining more than one sample from the same patient.

Another object of the invention is to provide a kit to assist in the diagnosing, differentiating and screening of an abnormal pregnancy, such as ectopic pregnancy, in a female patient using a marker that does not require measuring at different periods of gestation or obtaining more than one sample from the same patient.

### SUMMARY OF THE INVENTION

Serum Inhibin-A levels provide an important marker, with excellent statistical parameters, for screening and diagnosing pregnancies, or to differentiate among pregnancy abnormalities. The present invention features methods, kits and a test strip for differentiating and/or diagnosing normal and abnormal pregnancies in a female patient. The abnormal pregnancy is selected from, but is not limited to, an ectopic pregnancy, a threatened abortion, a spontaneous abortion, an incomplete abortion and a missed abortion. “Abnormal pregnancy”, for purposes of this invention, is a pregnancy that has a substantial chance of early (pre-third trimester) termination. Abnormal pregnancy does not include genetic defects of a fetus or a condition affecting the health of the mother, such as pre-emclapsia or toxemia. The method can be performed on a subject whether or not the patient has shown clinical manifestations of a pregnancy abnormality, and whether or not a separate laboratory test that indicates an abnormal pregnancy, such as an ectopic pregnancy, was performed on the subject.

The methods of differentiating and/or diagnosing an abnormal pregnancy, such as EP, includes obtaining a sample of a bodily fluid from the patient at about five to about ten weeks of gestation; determining the level of Inhibin-A in the sample; and comparing said level of Inhibin-A in said sample with a standard such as a physical standard. The determination of an abnormal pregnancy with the present inventive method can be performed by using only one sample from the patient.

The level of Inhibin-A to be determined in the present methods of differentiating and/or diagnosing can be levels of monomeric Inhibin-A, dimeric Inhibin-A or combinations thereof. The standard can be any physical standard, such as a known amount of Inhibin-A, but a statistical value may be used as the standard. The standard can be, but is not limited to, a value based on the average level of Inhibin-A in a normal pregnant female population. However, the standard should not be a sampling previously obtained from the same patient.

In the present invention, the sample of the patient to be tested can be a bodily fluid such as, but is not limited to, urine, blood, plasma or serum obtained from the patient's blood. The patient may have been determined to be pregnant by using a separate assay prior to, or during, the use of the present method. The level of Inhibin-A can be measured using an immunochemical assay, such as an enzyme-linked immunoassay.

The present invention is further drawn to a kit for differentiating, diagnosing and screening normal and abnormal pregnancies in a female patient. The kit is useful in carrying out the aforementioned methods of differentiation and diagnosis. The kit can be used to screen an abnormal pregnancy, such as EP. The kit can include a device for obtaining a sample of a bodily fluid from the patient at, for example, about five to about ten weeks of gestation, and a test for determining the level of Inhibin-A in the patient's sample.

The patient can first be determined to be pregnant by using a separate test or a separate assay. The levels of Inhibin-A in the patient's sample can be measured by using gold, a fluorescence tag, an enzyme tag, a radiolabeled tag and/or an immunochemical assay, such as an enzyme-linked immunoassay (ELISA).

The device in the kit can be, but is not limited to, a collection cup for urine, a syringe that can be used to obtain a blood sample, a needle and/or a collection cup. The kit can further contain an instruction manual. The instructions in the manual can give statistical information and/or explain how to carry out the test.

The present invention is also drawn to a test strip for differentiating between a normal pregnancy and an abnormal pregnancy. The test strip is useful for carrying out the previously mentioned methods of differentiation and diagnosis. The test strip can have a membrane for analyzing a bodily fluid, a captured antibody bound to the membrane and a reporter antibody. The captured antibody and the reporter antibody are specific for Inhibin-A. The reporter antibody can have an indicator attached thereto.

The indicator in the strip can be, but is not limited to, gold, an enzyme tag, a fluorescence tag, a radiolabeled tag or any chemical that can be quantitatively measured in an instrument.

A test kit for determining levels of Inhibin-A can include the test strip and a standard containing a known level of Inhibin-A. The standard can be a physical value such as a value based on the average level of Inhibin-A in a normal pregnant female population. The test kit can further include an instruction manual that contains information on performing the test for determining the level of Inhibin-A.

The present invention thus provides methods, kits and a test strip that can be used to diagnose and screen between viable and non-viable pregnancies.

### BRIEF DESCRIPTION OF THE FIGURES

FIG. 1. is an illustration of a strip test configuration and a comparison between results expected for an ectopic pregnancy and for a normal pregnancy.

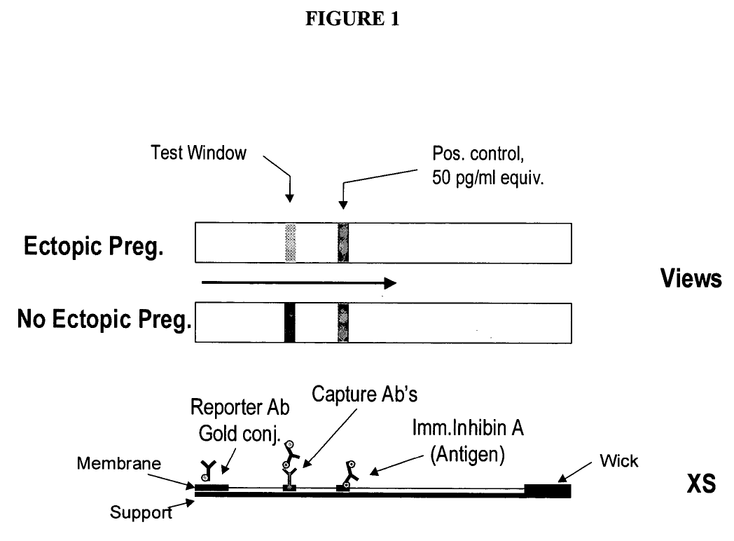

FIG. 2 is a chart showing Inhibin-A values of serum specimens from women with normal pregnancy (NORP), non-pregnant control (NPC) and 5 groups of abnormal pregnancy.

FIG. 3a. contains charts showing intra-group distribution of Inhibin-A levels for normal pregnancies (NORP) and non-pregnant controls (NPC).

FIG. 3b. contains charts showing intra-group distribution of Inhibin-A levels for ectopic pregnancies (EP) and missed abortions (MAB).

FIG. 4. is a graph depicting the stability of Inhibin-A in separated serum.

### DETAILED DESCRIPTION OF THE INVENTION

Abnormal pregnancies are recognized to be potentially hazardous to the patient's health and to the viability of the fetus. EP, for example, is a well known, life threatening medical condition affecting three in 100 pregnancies. The clinical symptoms are not specific and current laboratory tests are not sensitive to diagnosing EP. Laboratory tests, such as the dynamics of the hCG levels at 48 hours intervals, may suggest the diagnosis of EP but do not differentiate between EP and other abnormal pregnancies in early stages where hCG levels are <1500, due to ultrasound detecting an IUP occurring only when hCG levels are >1500.

Diagnosing an EP as early as possible is important in order to prevent potential complications. Early diagnosis may prevent life-threatening situations and allow the medical practitioner to offer medical therapy as early as possible to properly manage the pregnancy and/or patient's health. In addition, early diagnosis of EP may avoid surgical treatment and potential associated complications.

The present invention addresses these problems by providing methods, kits and a test strip that effectively uses Inhibin-A as a marker to differentiate and diagnose among normal and/or abnormal pregnancies. Particularly, Inhibin-A significantly differentiates between normal and abnormal pregnancies and between EP and other abnormal pregnancies. By providing a rapid diagnoses for EP, the present invention can provide the health care practitioner the information to efficiently manage the health and/or therapy for the patient.

The following data is of a study comparing hormone levels between EP and other pregnancy abnormalities, and between EP and non-pregnant controls. The study was undertaken in order to evaluate Inhibin-A as a rigorous diagnostic tool, to determine the predictive value of this hormone for ectopic pregnancy, and to investigate whether it can differentiate between ectopic pregnancy, and other types of abnormal pregnancy. The data reveals that a patient with a pregnancy abnormality, such as EP, presents typical non-pregnant hormonal levels of Inhibin-A.

### EXAMPLE

Methods:

Serum Inhibin-A in patients diagnosed with ectopic pregnancy (EP, n=17) by laparoscopy and pathology, was compared to levels in patients with normal pregnancy (NORP, n=40) miscarriage (MAB, n=35), incomplete abortion (INCAB, n=14), spontaneous abortion (SAB, n=5) and threatened abortion (THAB, n=6) and to non-pregnant controls (NPC, n=20).

Patients:

Pregnant patients were all in week 5-10 of gestation as assessed by first day of menses. The study group included patients (n=77) who presented with symptoms or clinical manifestations of abnormal pregnancy in the first trimester. Upon evaluation, they were further subdivided into the following: missed abortion (MAB, n=35), incomplete abortion (INCAB, n=14), spontaneous abortion (SAB, n=6), and threatened abortion (THAB, n=6). This group also included patients who, upon evaluation using laparoscopy and pathology, were diagnosed as having an ectopic pregnancy (n=17).

Two additional groups were used as controls. The non-pregnant Control (NPC, n=20) group included patients during reproductive age and with proven fertility, who applied for bilateral tubal ligation (BTL) sterilization. The Normal (no ovulation induction) Pregnancy Control group (NORP, n=40) were patients who presented for early normal pregnancy termination (elective pregnancy termination). Pregnancy in the latter group was confirmed at presentation by qualitative βhCG test, and product of conception was identified in the pathology report.

Specimens:

Serum was collected and frozen at −20 within 2 hrs of drawing.

Inhibin-A Assay:

Inhibin-A heterodimer was measured in serum using the ultrasensitive Oxford Bioinnovation (UK) kit, based on the two subunit-specific antibodies ELISA method described and validated. The kit has a sensitivity of 1.0 pg/ml, and is specific to Inhibin-A, with minimal cross-reactivity with the Proα(C) subunit, Inhibin-B, or Activins. Inter- and intra-plate variations are <10%.

Data Analysis:

ANOVA analysis was intended, yet the variances of key group in this study, as well as the bimodal distribution in more than one group precluded this approach. Data were analyzed by the rank-sum test (the Mann-Whitney U test).

Summary of Results:

Inhibin-A in EP was significantly lower than in NORP (12.7±11.7 and 237.3±125.9 pg/ml, p<<0.0002), however similar to NPC (12.7±11.7 and 13.5±14.2 pg/ml, p=0.43). Inhibin-A levels in the abnormal pregnancies were significantly lower than in the NORP group: MAB 42.4±54.9 pg/ml (p<<0.0002), and SAB 47.5±55.6 pg/ml (p<<0.0002) and INCAB 12.2±10.5 pg/ml (p<<0.0002) but not statistically significant in THAB 183.1±119.4 pg/ml (p=0.159).

Analysis of Results

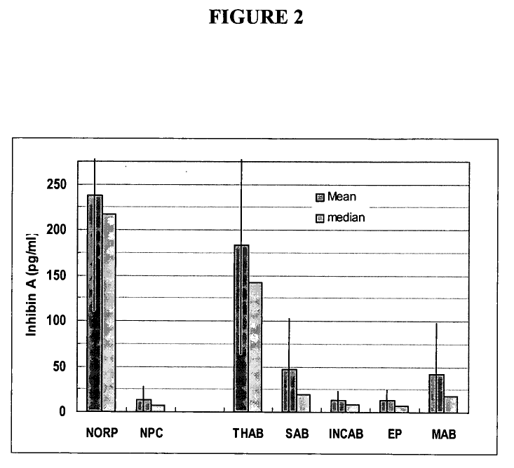

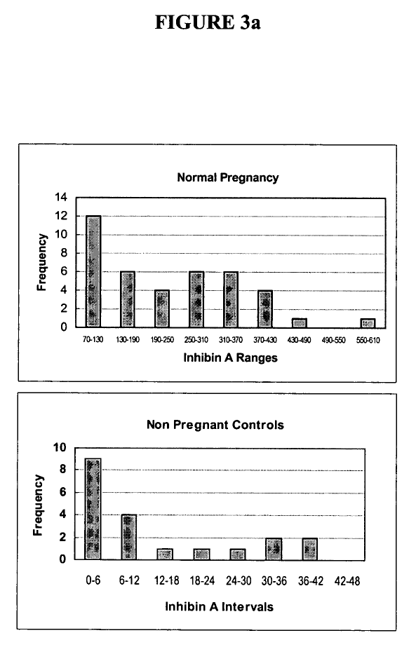

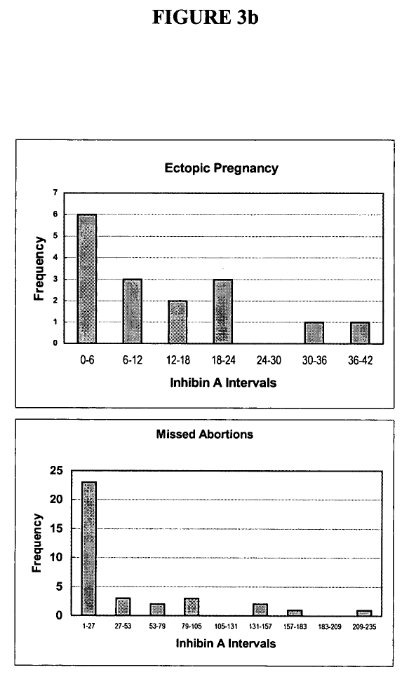

The chart in FIG. 2 shows (left side) the full range of differences between values of normal pregnant women (NORP) and those of non-pregnant controls (NPC). FIG. 2 also depicts values (right side) from the five different groups of patients assessed with different types of abnormal pregnancy. Dark and light columns represent the means and the medians respectively, vertical bars are +/−one standard deviation. In general, Inhibin-A values in the abnormal pregnancies are lower than the values of normal pregnancy and in some cases resemble those of normal non-pregnant controls. Because in most groups the standard deviation was large, and the median was consistently lower than the mean, it is believed that a non-normal, non-poissonic distribution is the cause. None of the groups examined show a normal distribution (depicted in FIG. 3a and FIG. 3b). In all cases, two subpopulations were apparent. Therefore, the intra-group distribution of Inhibin-A values in all groups were examined with greater than ten cases examined per group (FIG. 3a and FIG. 3b)

The histograms in FIGS. 3a and 3b reveal distributions that can not readily be defined as normal or poissonic. The full range of values within each group was divided in 7-9 bins to best depict the deviation from a normal distribution of values. Some key abnormal pregnancy groups in FIGS. 3a and 3b are demonstrative of a non-Gaussian distribution. The distribution shows at least two subpopulations in each histogram. It is not completely clear what causes this phenomenon. No apparent clinical basis was associated with these differences (between subgroups), as patients did not report differences in clinical symptoms.

Likewise, no correlation was found within gestation weeks 5-10 between the Inhibin-A values and the week of gestation (r²=0.23 in EP and r²=0.17 in NORP). In the normal pregnancy (NORP) chart, two groups show up: one group with Inhibin-A concentration of 70-190 pg/ml and one with 250-400 pg/m. In the non-pregnant control (NPC), the majority of patients had values in the 0-12 pg/ml range with a small group characterized by values of 24-42 pg/ml. Within the EP group, it was found that more than 50% of the specimens' Inhibin-A values are between 0-18 pg/ml, much lower than that of a typical luteal phase. Only two cases had values in the 30-40 pg/ml range. Lastly, in the missed abortion group (MAB), most patients appear to have very low values as well, however, there were scattered cases with higher values, as high as 230 pg/ml that are well within the 70-250 pg/ml range of the normal subpopulation. Notably, under these circumstances we could not perform statistics with the commonly selected tests of ANOVA or the t-test, and resorted to the rank-sum or Mann-Whitney U test.

TABLE 1
P-Values in Mann-Whitney tests

| | NORP | NPC | EP |
|---|---|---|---|
| NORP | X | | |
| NPC | <<0.0002 | X | |
| EP | <<0.0002 | 0.430 | X |
| THAB | 0.159 | <<0.0002 | 0.016 |
| SAB | <<0.0002 | 0.325 | 0.560 |
| INCAB | <<0.0002 | 0.345 | 0.310 |
| MAB | <<0.0002 | 0.006 | 0.011 |

Table 1 depicts the statistical significance (P values) of differences in the Inhibin-A levels between the various study and control groups. This test confirmed the overall observation of FIG. 2, suggesting that with the exception of threatened abortion (THAB), all other forms of abnormal pregnancy cases encountered in this study had significantly lower Inhibin-A values as compared to Inhibin-A values in the normal pregnancy groups. An apparent mirror image is found when comparing the Inhibin-A values in the non-pregnant controls (NPC) with the NORP groups. The abnormal pregnancy groups significantly differed (p<<0.05) relative to NORP values but are similar (P≧0.05) to NPC and vice versa. MAB values were the only exception to this rule, and were found to be significantly higher than the NPC group. The value of Inhibin-A in EP was indistinguishable from NPC in this study (p=0.43), probably due to the distribution of NPC's that were skewed to follicular phase patients. The values obtained for the levels of Inhibin-A in EP patients were significantly lower than in MAB and THAB patients.

Regarding the abnormal distribution of Inhibin-A values in EP, normally Inhibin-A levels are controlled during the first few weeks of gestation by the luteal follicle. This suggests that serum levels in early pregnancy should be at the level of normal cycle luteal phase. However, it is known that luteal ovarian functions (secretions) are significantly suboptimal in patients with EP. These suboptimal functions have originally been observed with very low progesterone levels as well as other luteal ovarian markers. With this notion, and the bimodal distribution, the data can perhaps be indicating heterogeneity in the etiology of EP. While the majority of patients have low values, potentially consequential or related to a hypoactive luteal ovary, the few cases with the higher values of Inhibin-A in the EP group may have a different etiology(ies) unrelated to low hormonal activity of the luteal ovary (e.g. anatomical, cilliary motion-related etc, infections etc). In either case, all Inhibin-A values in EP are significantly lower than in all other viable pregnancies.

TABLE 2

| Decisions between EP and: | NORP | THAB | NORP | THAB |
|---|---|---|---|---|
| Cutoff value: | 45 | 45 | 36 | 36 |
| False positive rate | 0.0 | 0.0 | 0.0 | 0.0 |
| False negative rate | 0.0 | 0.0 | 5.0 | 5.0 |
| Sensitivity | 100.0 | 100.0 | 95.0 | 95.0 |
| Specificity | 100.0 | 100.0 | 100.0 | 100.0 |
| Accuracy | 100.0 | 100.0 | 98.4 | 96.2 |
| PPV | 100.0 | 100.0 | 100.0 | 100.0 |
| NPV | 100.0 | 100.0 | 97.6 | 85.7 |
| Odds Ratio | infinite | infinite | infinite | infinite |

Table 2 depicts the statistical parameters in the prediction of ectopic pregnancy using Inhibin-A testing. The distribution of data points shows a complete separation between Inhibin-A values in the ectopic pregnancy and the normal pregnancy groups. Namely, the highest value of EP is well below the lowest value in the normal pregnancy group. This obviates an excellent statistics as specified in Table 2. The gap between these values, being 30 pg/ml units, creates a wide range to place cut-off values that yields 100% sensitivity, specificity, accuracy, PPV (positive predictive value) and NPV (negative predictive value), with no anticipation of both the positive and negative false rate. Table 2 shows the statistical parameters for a cut-off value of 45 pg/ml and 36 pg/ml. Threatened abortion was found in FIG. 2 to be lower than NORP, but with insignificant difference. The above statistical parameters between EP and THAB were compared as well, showing an excellent predictive (differentiating) power in this pair as well.

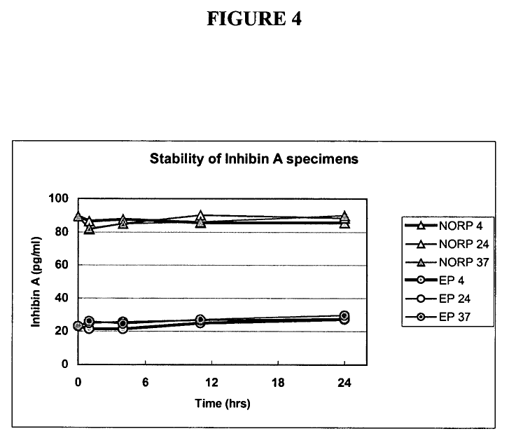

Upon finding that the Inhibin-A values in EP were the lowest of all groups, and considering that measurement of this analyte for diagnostic or confirmatory purposes may be done on specimens of several hours old following phlebotomy, the stability of serum specimens from normal and EP patients at three storage temperatures for 24 hrs was evaluated. This was done with the aim of establishing whether or not such lower values reflect exceptionally faster than normal degradation of immunoreactive Inhibin-A in EP patients. FIG. 4 is a graph depicting the stability of Inhibin-A in separated serum. Two specimens (one from EP and one from NORP) were collected and separated and frozen in aliquots within less than one hour. Aliquots were thawed at different times (1, 4, 10 and 24 hr.) before testing. As shown in FIG. 4, Inhibin-A specimens are stable up to 24 hrs, both in normal and EP specimens, and no enhanced deterioration could possibly explain the low values in EP. The lack of difference at the three temperatures suggests that no enzymatic process is affecting changes in the hormone levels during storage as a separated serum specimen. Therefore, these measurements reflect true values rather than a technical flaw.

### CONCLUSION

The results obtained from the above experiment demonstrate that Inhibin-A levels in early pregnancy (week 5-10) can be used as a stand-alone marker (not requiring βhCG or any additional marker for decision making, and not requiring a 48 h waiting period to assess βhCG) for the following applications:
1. Diagnosis/confirmation of a pregnancy abnormality, such as EP, in patients having non-specific clinical symptoms.
2. Differentiating between an abnormal pregnancy, such as EP, and viable pregnancies (THAB, NORP).
3. Differentiating between an abnormal pregnancy, such as EP, and other non-viable pregnancies (MAB, SAB)
4. Screening and detection of a pregnancy abnormality, such as EP, before the manifestation of any clinical non-specific symptoms.

Consequently, serum Inhibin-A levels provide an important marker, with excellent statistical parameters, for the diagnosis and management of a pregnancy abnormality and to differentiate the abnormal pregnancy from other abnormal pregnancies.

The foregoing description of the specific embodiments will so fully reveal the general nature of the present invention that others can, by applying current knowledge, readily modify and/or adapt for various applications such specific embodiments without departing from the generic concepts, and therefore such adaptations and modifications are intended to be comprehended within the meaning and range of equivalents of the disclosed embodiments. It is to be understood that the phraseology or terminology herein is for the purpose of description and not limitation.

All references cited in this specification are hereby incorporated by reference in their entirety.

What is claimed is:

1. A method of differentiating between a normal pregnancy and an abnormal pregnancy in a female patient, comprising:
a) obtaining a sample of a bodily fluid from the patient at about five to about ten weeks of gestation;
b) determining the level of Inhibin-A in said sample; and
c) comparing said level of Inhibin-A in said sample with a standard.

2. The method of claim 1, wherein said abnormal pregnancy is selected from the group consisting of a threatened abortion, a spontaneous abortion, an incomplete abortion, an ectopic pregnancy and a missed abortion.

3. The method of claim 2, wherein said abnormal pregnancy is an ectopic pregnancy.

4. The method of claim 3, wherein said patient has manifested clinical symptoms of ectopic pregnancy.

5. The method of claim 4, wherein said clinical symptoms were manifested through a separate laboratory test that indicates ectopic pregnancy.

6. The method of claim 4, wherein said clinical symptoms were manifested without subjecting said patient to a separate laboratory test that indicates ectopic pregnancy.

7. The method of claim 3, wherein said patient has not manifested clinical symptoms of ectopic pregnancy.

8. The method of claim 7, wherein the patient was subjected to a separate laboratory test that indicates ectopic pregnancy.

9. The method of claim 1, wherein said Inhibin-A is selected from the group consisting of monomeric Inhibin-A, dimeric Inhibin-A and combinations thereof.

10. The method of claim 1, wherein said standard is a physical standard containing a known amount of Inhibin-A.

11. The method of claim 10, wherein said standard is a value based on the average level of Inhibin-A in a normal pregnant female population.

12. The method of claim 1, wherein said bodily fluid is selected from the group consisting of urine, blood, plasma and serum.

13. The method of claim 1, wherein said level of Inhibin-A in said sample is measured using an immunochemical assay.

14. The method of claim 13, wherein said immunochemical assay is an enzyme-linked immunoassay.

15. A method of diagnosing an abnormal pregnancy in a female patient comprising:
a) obtaining a sample of a bodily fluid from the patient at about five to about ten weeks of gestation;
b) determining the level of Inhibin-A in said sample; and
c) comparing said level of Inhibin-A in said sample with a standard.

16. The method of claim 15, wherein said abnormal pregnancy is selected from the group consisting of a threatened abortion, a spontaneous abortion, an incomplete abortion, an ectopic pregnancy and a missed abortion.

17. The method of claim 16, wherein said abnormal pregnancy is an ectopic pregnancy.

18. The method of claim 15, wherein said Inhibin-A is selected from the group consisting of monomeric Inhibin-A, dimeric Inhibin-A and combinations thereof.

19. The method of claim 15, wherein said standard is a physical standard containing a known amount of Inhibin-A.

20. The method of claim 19, wherein said standard is a value based on the average level of Inhibin-A in a normal pregnant female population.

21. The method of claim 15, wherein said bodily fluid is selected from the group consisting of urine, blood, plasma and serum.

22. The method of claim 15, wherein said level of Inhibin-A in said test is determined using an immunochemical assay.

23. The method of claim 22, wherein the patient is determined to be pregnant by a separate assay.

24. A kit for differentiating between a normal pregnancy and an abnormal pregnancy in a female patient comprising:
a) a device for obtaining a sample of a bodily fluid from the patient at about five to about ten weeks of gestation; and
b) a test for determining the level of Inhibin-A in said sample.

25. The kit of claim 24, wherein said abnormal pregnancy is selected from the group consisting of a threatened abortion, a spontaneous abortion, an incomplete abortion, an ectopic pregnancy and a missed abortion.

26. The kit of claim 25, wherein said abnormal pregnancy is an ectopic pregnancy.

27. The kit of claim 24, wherein the test comprises comparing the level of Inhibin-A obtained from said sample with a standard.

28. The kit of claim 27, wherein said standard is a physical standard containing a known amount of Inhibin-A.

29. The kit of claim 28, wherein said standard is a value based on the average level of Inhibin-A in a normal pregnant female population.

30. The kit of claim 24, wherein said test is selected from the group consisting of tests for determining levels of monomeric Inhibin-A, dimeric Inhibin-A and combinations thereof.

31. The kit of claim 24, wherein said bodily fluid is selected from the group consisting of urine, blood, plasma and serum.

32. The kit of claim 24, wherein said level of Inhibin-A in said sample is measured using an immunochemical assay.

33. The kit of claim 32, wherein said immunochemical assay is an enzyme-linked immunoassay.

34. The kit of claim 24, further comprising an additional test for determining whether the patient is pregnant.

35. The kit of claim 24, wherein the device is selected from the group consisting of a syringe, a needle, a collection cup and combinations thereof.

36. A test strip for differentiating between a normal pregnancy and an abnormal pregnancy comprising:
a) a membrane for analyzing a bodily fluid;
b) a capture antibody bound to the said membrane, said capture antibody being specific for Inhibin-A; and
c) a reporter antibody, said reporter antibody being specific for Inhibin-A and having an indicator attached thereto.

37. The test strip of claim 36, wherein said abnormal pregnancy is selected from the group consisting of a threatened abortion, a spontaneous abortion, an incomplete abortion, an ectopic pregnancy and a missed abortion.

38. The test strip of claim 37, wherein said abnormal pregnancy is an ectopic pregnancy.

39. The test strip of claim 36, wherein said Inhibin-A is selected from the group consisting of monomeric Inhibin-A, dimeric Inhibin-A and combinations thereof.

40. The test strip of claim 36, wherein said indicator is gold.

41. A test kit comprising the test strip of claim 36 and a standard containing a known level of Inhibin-A.

42. The test kit of claim 41, wherein said standard is a value based on the average level of Inhibin-A in a normal pregnant female population.

43. The test kit of claim 41 further comprising an instruction manual.

44. The test kit of claim 43, wherein said instruction manual contains information on performing said test for determining the level of Inhibin-A.
